(19) United States
(12) Patent Application Publication
(10) Pub. No.: US 2026/0240605 A1
(43) Pub. Date: Aug. 20, 2026

(54) ULTRASOUND AND MAGNETIC FIELD USED WITH MAGNETO-ELASTIC MATERIAL FOR DETERMINNG TISSUE ENGAGEMENT BY MEDICAL DEVICE

(71) Applicant: KONINKLIJKE PHILIPS N.V., EINDHOVEN (NL)

(72) Inventor: Thomas John MCPEAK, CALEDONIA, MN (US)

(21) Appl. No.: 19/471,132

(22) PCT Filed: Apr. 24, 2024
(86) PCT No.: PCT/EP2024/061171

§ 371 Date: Sep. 30, 2025

Related U.S. Application Data
(60) Provisional application No. 63/464,320, filed on May 5, 2023.

(51) Int. Cl. A61B 34/20; A61B 17/00

(52) U.S. Cl. CPC A61B 34/20; A61B 17/00; A61B 2017/00039; A61B 2017/00106; A61B 2017/00115; A61B 2034/2051; A61B 2034/2063

(57) ABSTRACT

A system includes a magnetic field subsystem configured to generate an oscillating magnetic field and a magneto-elastically driven oscillator configured to, in the presence of the oscillating magnetic field, generate acoustic waves. The system also includes an ultrasound probe configured to receive the generated acoustic waves and generate an electrical signal indicative of the generated acoustic waves. A processing circuit is configured to output, to a display, a graphical representation indicative of the generated acoustic waves. Contact between the magneto-elastically driven oscillator and tissue of a patient causes a change in the generated acoustic waves, the generated electrical signal, and the graphical representation.

### TECHNICAL FIELD

The subject matter described herein relates to a device, systems, and methods for detecting and measuring tissue engagement by medical devices. The degree or extent that the medical device contacts tissue can be determined using magneto-elastic material with ultrasound and magnetic field.

### BACKGROUND

For many medical procedures (e.g., edge-to-edge heart valve repair, heart valve cord repair, etc.), it is important to ensure that the proper amount of tissue is being engaged by a medical device (e.g., a clip, tissue anchor, cutting or ablation tool, etc.) in order to hold the device in place, to provide the proper amount of tension without the device pulling free of tissue anchorage, or to cut through tissue growths or obstructions.

Existing tools provide no good way to ensure that the proper amount of tissue is engaged, other than visually verifying via a mark or stop on the device being inserted. However, direct visual verification is not always possible. For example, in some cases the only indications may come from fluoroscopic or ultrasonic imaging of the tissue and device. However, especially with fluoroscopic imaging, accurately discerning the amount of tissue engaged is difficult. Also, with ultrasonic imaging, shadowing, especially with metallic implants, can make evaluation of engagement difficult. Other tissue engagement sensing methods are active and require a power source and/or an electrical connection from which to capture a signal.

It should therefore be appreciated that such commonly used tissue engagement sensing methods have numerous drawbacks, including poor accuracy and repeatability.

The information included in this Background section of the specification, including any references cited herein and any description or discussion thereof, is included for technical reference purposes only and is not to be regarded as subject matter by which the scope of the disclosure is to be bound.

### SUMMARY

Disclosed is a tissue engagement sensing system. The tissue engagement sensing system disclosed herein has particular, but not exclusive, utility for verifying accurate placement of implantable devices such as heart valve clips and tissue anchors. The system includes a wireless micro-electromechanical systems (MEMS) based sensor that can be attached to any fluid-surrounded instrument to detect the depth of the sensor's and/or instrument's insertion into tissue. The MEMS based sensor includes a magneto-elastic material that change dimension when exposed to a magnetic field. The magneto-elastic material is employed to determine the length of tissue that impinges on the surface of the sensor, allowing for a wireless, non-optical determination of tissue engagement. With a comb-like construction, the sensor includes a number of magneto-elastically driven teeth or spars projecting from a support member. An oscillating magnetic field will therefore cause the teeth to vibrate, unless they are blocked by contact with tissue, and these vibrations can be measured by an ultrasound probe to determine the number of teeth that are engaged with the tissue, and hence the amount or degree of penetration or engagement of the instrument.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system. The system includes a magnetic field subsystem configured to generate an oscillating magnetic field; a magneto-elastically driven oscillator configured to, in the presence of the oscillating magnetic field, generate acoustic waves; an ultrasound probe configured to receive the generated acoustic waves and generate an electrical signal indicative of the generated acoustic waves; and a processing circuit configured to output, to a display, a graphical representation indicative of the generated acoustic waves, where contact between the magneto-elastically driven oscillator and tissue of a patient causes a change in the generated acoustic waves, the generated electrical signal, and the graphical representation. Other embodiments of this aspect include corresponding computer systems, apparatus, and computer programs recorded on one or more computer storage devices, each configured to perform the actions of the methods.

Implementations may include one or more of the following features. In some aspects, the system may include: a second magneto-elastically driven oscillator configured to, in the presence of the oscillating magnetic field, generate the acoustic waves; and a support member, where the magneto-elastically driven oscillator and the second magneto-elastically driven oscillator extend from the support member, where at least one of a frequency or an amplitude of the generated acoustic waves is indicative of contact between the tissue and the magneto-elastically driven oscillator or the second magneto-elastically driven oscillator, such that the graphical representation is indicative of a degree of engagement of the tissue by the magneto-elastically driven oscillator and the second magneto-elastically driven oscillator. In some aspects, the support member is coupled to a tissue-engaging portion of the tissue-engaging instrument, such that the graphical representation is indicative of a degree of engagement of the tissue by the tissue-engaging portion. In some aspects, the system may include a plurality of additional magneto-elastically driven oscillators extending from the support member and configured to, in the presence of the oscillating magnetic field, generate the acoustic waves. In some aspects, the support member may include a first material, and where each magneto-elastic oscillator may include a tooth may include the first material. In some aspects, each magneto-elastic oscillator further may include a magneto-elastic material different from the first material. A first end of each magneto-elastic oscillator is coupled to the support member and a second end of each magneto-elastic oscillator is coupled to the second support member. In some aspects, the magneto-elastic oscillator is configured to be at least partially immersed in a fluid, where the generated acoustic waves propagate through the fluid, and where the ultrasound probe is configured to receive the generated acoustic waves through at least the fluid. In some aspects, the ultrasound probe is further configured to generate second acoustic wave, where the magneto-elastic oscillator is further configured to, in the presence of the second acoustic waves, generate a second oscillating magnetic field; where the system further may include a magnetic field subsystem configured to detect a location of the magneto-elastic oscillator associated with the second oscillating magnetic field and generate a corresponding location signal; and a processing circuit configured to receive the location signal and output, to a display, a second graphical representation indicative of the location signal. Implementations of the described techniques may include hardware, a method or process, or computer software on a computer-accessible medium.

In some aspects, the system further includes a plurality of magneto-elastically driven oscillators, where the plurality of magneto-elastically driven oscillators include the magneto-elastically driven oscillator, where the graphical representation is indicative of a degree of engagement of the tissue by the tissue-engaging portion, and where the processing circuit is configured to determine the degree of engagement based on a quantity of the plurality of magneto-elastically driven oscillators that are in contact with the tissue. In some aspects, the change in the generated acoustic waves includes a decrease in an amplitude of the generated acoustic waves. In some aspects, the system further includes a plurality of magneto-elastically driven oscillators, where the plurality of magneto-elastically drive oscillators includes the magneto-elastically driven oscillator, where the graphical representation is indicative of a degree of engagement with the tissue, and where the processing circuit is configured to determine the degree of engagement based on a quantity of the plurality of magneto-elastically driven oscillators that are not in contact with the tissue. In some aspects, the magneto-elastically driven oscillator includes a microelectromechanical systems (MEMS) spring. In some aspects, a change in the generated acoustic waves includes an increase in an amplitude of the generated acoustic waves. In some aspects, a magneto-strictive response of the magneto-elastically driven oscillator is configured to increase in response to a mechanical strain caused by contact with the tissue.

One general aspect includes a system. The system includes an ultrasound probe configured to generate acoustic waves; a magneto-elastically driven oscillator configured to, in the presence of the acoustic waves, generate an oscillating magnetic field; a magnetic field subsystem configured to detect a location of the magneto-elastically driven oscillator associated with the oscillating magnetic field and generate a corresponding location signal; and a processing circuit configured to receive the location signal and output, to a display, a first graphical representation indicative of the location signal. Other embodiments of this aspect include corresponding computer systems, apparatus, and computer programs recorded on one or more computer storage devices, each configured to perform the actions of the methods.

Implementations may include one or more of the following features. In some aspects, the system may include a medical instrument, where the magneto-elastic oscillator is coupled to a portion of the medical instrument, such that the first graphical representation is indicative of a location of the portion. In some aspects, the support member is coupled to a tissue-engaging portion of the tissue-engaging instrument, such that the second graphical representation is indicative of a degree of engagement of the tissue by the tissue-engaging portion. In some aspects, the magneto-elastically driven oscillator and the second magneto-elastically driven oscillator extend from the support member. In some aspects, the system may include a plurality of additional magneto-elastically driven oscillators extending from the support member and configured to, in the presence of the second oscillating magnetic field, generate the oscillating magnetic field. In some aspects, the support member may include a first material, and where each magneto-elastic oscillator may include a tooth may include the first material. In some aspects, each magneto-elastic oscillator further may include a magneto-elastic material different from the first material. In some aspects, at least one of a frequency or an amplitude of the oscillating magnetic field is indicative of contact between the tissue and the magneto-elastically driven oscillator or the second magneto-elastically driven oscillator, where the processing circuit is further configured to generate a second graphical representation indicative of a degree of engagement of the tissue by the magneto-elastically driven oscillator or the second magneto-elastically driven oscillator. Implementations of the described techniques may include hardware, a method or process, or computer software on a computer-accessible medium.

In some aspects, a magneto-strictive response of the magneto-elastically driven oscillator is configured to increase in response to a mechanical strain caused by contact with tissue of a patient. In some aspects, an amplitude of the oscillating magnetic field is configured to increase in response to the increase in the magneto-strictive response. In some aspects, the magneto-elastically driven oscillator includes a microelectromechanical systems (MEMS) spring.

One general aspect includes a system a sensor may include: a support structure; a plurality of teeth extending from the support structure; and a plurality of corresponding magneto-elastic members coupled to the plurality of teeth. The system also includes where: an excitation of the plurality of teeth by first acoustic waves causes the plurality of magneto-elastic members to generate a first oscillating magnetic field detectable by a magnetic field detector, an excitation of the plurality of magneto-elastic members by a second oscillating magnetic field causes the plurality of teeth to generate second acoustic waves detectable by an ultrasound probe, an engagement of one or more of the teeth by tissue of a patient changes at least one of an amplitude or a frequency of the first oscillating magnetic field and of the second acoustic waves by an amount corresponding to a number of the teeth that are engaged by the tissue. Other embodiments of this aspect include corresponding computer systems, apparatus, and computer programs recorded on one or more computer storage devices, each configured to perform the actions of the methods.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the tissue engagement sensing system, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a schematic representation of at least a portion of an example tissue engagement sensing system, in accordance with at least one embodiment of the present disclosure.

FIG. 2 is a schematic representation, in block diagram form, of at least a portion of an example tissue engagement sensing system, in accordance with at least one embodiment of the present disclosure.

FIG. 3A is a schematic representation of an exemplary magneto-elastic member, in accordance with at least one embodiment of the present disclosure.

FIG. 3B is a schematic representation of the exemplary magneto-elastic member of FIG. 3A, in accordance with at least one embodiment of the present disclosure.

FIG. 4A is a schematic, diagrammatic bottom view of an exemplary tissue engagement sensor, in accordance with at least one embodiment of the present disclosure.

FIG. 4B is a schematic, diagrammatic bottom view of an exemplary tissue engagement sensor, in accordance with at least one embodiment of the present disclosure.

FIG. 5 is a schematic, diagrammatic side view of an exemplary tissue engagement sensor, in accordance with at least one embodiment of the present disclosure.

FIG. 6 is a schematic, diagrammatic side view of an exemplary tissue engagement sensor, in accordance with at least one embodiment of the present disclosure.

FIG. 7 is a schematic, diagrammatic representation, in block diagram form, of at least a portion of an example tissue engagement sensing system, in accordance with at least one embodiment of the present disclosure.

FIG. 8 is a schematic representation, in block diagram form, of at least a portion of an example instrument tracking system employing components of the tissue engagement sensing system, in accordance with at least one embodiment of the present disclosure.

FIG. 9 is a schematic diagram of a processor circuit, in accordance with at least one embodiment of the present disclosure.

FIG. 10 is a schematic, diagrammatic top view of an exemplary tissue engagement sensor, in accordance with at least one embodiment of the present disclosure.

FIG. 11 is a schematic, diagrammatic side cross-sectional view of an exemplary tissue engagement sensor of FIG. 10, in accordance with at least one embodiment of the present disclosure.

FIG. 12 is a schematic, diagrammatic front cross-sectional view of an exemplary tissue engagement sensor of FIG. 10, in accordance with at least one embodiment of the present disclosure.

FIG. 13 is a graph showing magnetostriction or magneto-strictive response of a magneto-elastic material as a function of the strength of the variable (alternating) magnetic field, in accordance with at least one embodiment of the present disclosure.

FIG. 14 is a schematic representation, in block diagram form, of an example tissue engagement sensing system, in accordance with at least one embodiment of the present disclosure.

FIG. 15 is a schematic, diagrammatic side view of an exemplary tissue engagement sensor, in accordance with at least one embodiment of the present disclosure.

FIG. 16 is a is a schematic, diagrammatic side view of the exemplary tissue engagement sensor of FIG. 15, loaded with a mechanical strain and/or a constant (bias) magnetic field, in accordance with at least one embodiment of the present disclosure.

FIG. 17A is a schematic, diagrammatic bottom view of an exemplary tissue engagement sensor, in accordance with at least one embodiment of the present disclosure.

FIG. 17B is a schematic, diagrammatic front view of the exemplary tissue engagement sensor of FIG. 17A, in accordance with at least one embodiment of the present disclosure.

FIG. 18 is a schematic representation, in block diagram form, of at least a portion of the example instrument tracking system employing components of the tissue engagement sensing system, in accordance with at least one embodiment of the present disclosure.

### DETAILED DESCRIPTION

In accordance with at least one embodiment of the present disclosure, a tissue engagement sensing system is provided which includes a wireless microelectromechanical (MEMS) based sensor that can detect the distance a device is inserted into tissue or the distance tissue is inserted into a device. The device includes enough space to attach the MEMS sensor, and the tissue and sensor can be surrounded by fluid (a heart chamber, inside a blood vessel, or inside the bladder, etc.) through which acoustic waves can propagate.

Magneto-elastic materials have the unique property of changing dimension when exposed to a magnetic field, much like piezoelectric crystalline materials change dimension when exposed to an electric field. Disclosed herein is a sensor that can determine the length of tissue that impinges on the surface of the sensor, allowing for a wireless, non-optical method of determining tissue engagement.

One embodiment uses a comb-like construction, with a number of teeth or spars projecting from a support member. In some instances, these teeth may also be referred to as beams or arms. Each tooth includes a small magneto-elastic pusher, designed to change in length when a magnetic field is applied. This change in length causes the tooth to flex in a direction perpendicular to the length of the support member. If the magnetic field is fluctuating/in free space, this flexing of the tooth will cause the tooth to vibrate at a frequency consistent with the magnetic field fluctuations. However, if the tooth is in contact with tissue, this will retard or dampen the vibration of that individual tooth. If the magnetic field is fluctuating at a frequency in the range detectable by an ultrasonic imaging system, the vibrations of the teeth will effectively become an ultrasound emitter, with the pressure of the acoustic waves generated (and hence the brightness of the MEMS tissue engagement sensor in the ultrasound image) corresponding to (e.g., proportionally or non-proportionally related to) to the number of teeth that are vibrating.

In an example, the signal from the MEMS sensor is measured prior to insertion of a medical instrument into tissue, or prior to grasping tissue. The medical instrument may for example be an intrabody instrument, intracavity instrument, intraluminal instrument, or other type of medical instrument. Since no tissue is blocking the vibration of the teeth, a baseline (e.g., maximum amplitude or brightness) signal can be measured. Then, after the instrument engages the tissue, the difference in the signal strength vs. the baseline signal should correspond or be proportional to the number of teeth that are being damped, and consequently, the length of tissue that impinges on the MEMS tissue engagement sensor.

This process alleviates the need for direct visual verification of the depth to which an instrument is inserted into tissue or grabs onto tissue. Furthermore, while most tissue engagement sensing methods are active, and require a power source and/or an electrical connection to the sensor, the MEMS sensor disclosed herein can be completely passive, and is (at least in principle) detectable by any ultrasound imaging system, including but not limited to transesophageal echocardiography (TEE), transthoracic echocardiography (TTE), or intracardiac echocardiography (ICE) systems. When incorporated onto the surface of an implant, the MEMS sensor can also be interrogated over time, to determine if the amount of tissue engaged is changing between measurements.

Another advantage of the present disclosure is that the MEMS tissue engagement sensor can be applied retroactively to any device, without requiring any electrical connections that could interfere with the operation of the device. Furthermore, the MEMS tissue engagement sensor could be applied to any location on the device that has enough surface area for it. It is noted that the MEMS sensor can be fabricated in a variety of different sizes, shapes, numbers of teeth, etc., to maximize its utility for different applications.

Thus, an aspect of this invention is the MEMS tissue engagement sensor itself, which may for example be constructed of a base material with a longitudinal well, over which a number of magneto-elastically driven teeth are cantilevered. The sensor could be attached to a medical device or implant with the well parallel to the dimension of tissue engagement, such that progressive engagement of more tissue results in more teeth of the MEMS sensor being damped, which then generates a weaker ultrasound signal return. In an example, the ultrasound imaging system may be capable of detecting vibrations at frequencies of 4 MHz and below, since 4 MHz may be close to the upper limit of a magneto-elastic material's ability to change dimension effectively. There are a number of shapes that would lend themselves to making the comb and tooth members, and the members could be cantilevered (e.g., free on one end) or could be anchored at both ends. The sensor can also be run in reverse-stimulated with ultrasound waves in order to produce an oscillating magnetic field that can be located with a magnetic field sensor. Thus, the sensor can be part of a medical instrument location system, a tissue engagement sensing system, or a combined medical instrument location and tissue engagement sensing system.

The present disclosure aids substantially in precisely measuring tissue engagement by a medical device or implant, with resolution as large as several millimeters and as small as 100 microns, depending on the spacing of the teeth. Other resolutions, both larger and smaller, may be used instead or in addition. The MEMS tissue engagement sensor provides a clearly detectable signal whose strength corresponds to or is proportional to the amount of tissue engaged. Implemented as a MEMS device that is excited by an oscillating magnetic field or an alternating magnetic field (e.g., generated by an alternating current) and measured by an ultrasound imaging system, the tissue engagement sensing system disclosed herein provides a practical means for wirelessly measuring tissue engagement. This improved tissue engagement sensing ability transforms a process based largely on image interpretation and guesswork into one that is precise and repeatable, without the normally routine need to interrogate a wired sensor or visually inspect a stop or guide mark on the device. This unconventional approach improves the functioning of the medical device or implant, by ensuring proper placement and tissue engagement.

These descriptions are provided for exemplary purposes only, and should not be considered to limit the scope of the tissue engagement sensing system. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

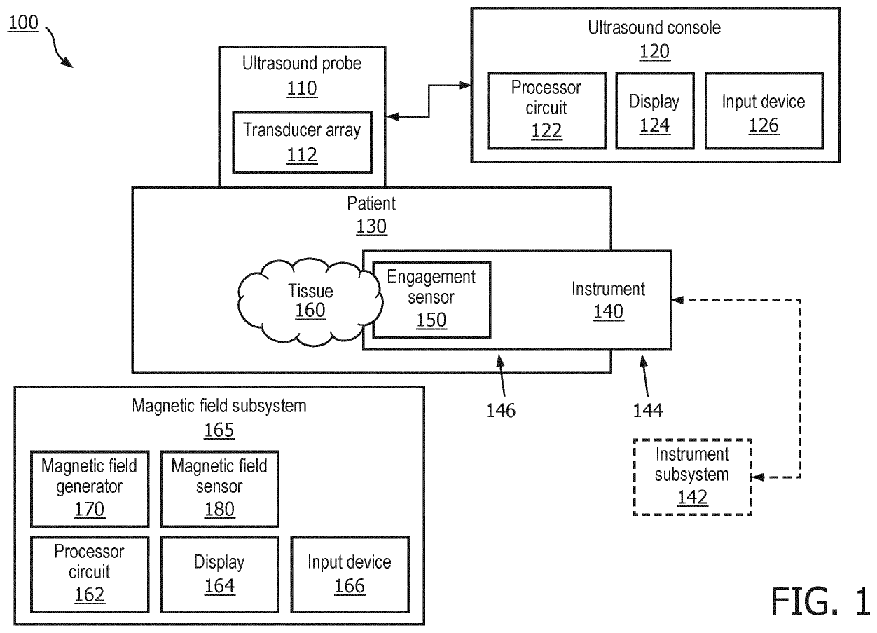

FIG. 1 is a schematic representation of at least a portion of an example tissue engagement sensing system 100, in accordance with at least one embodiment of the present disclosure. In the example shown in FIG. 1, the tissue engagement sensing system 100 includes an ultrasound probe 110 which includes a transducer array 112 and is in contact with a patient 130. The ultrasound probe 110 can be an external probe, or can be an intraluminal probe such as an intracardiac echography (ICE), trans-esophageal echocardiogram (TEE), or intravascular ultrasound (IVUS) probe.

The tissue engagement sensing system 100 also includes an ultrasound console 120, which includes a processor circuit 122, display 124, and input device 126. An instrument 140 is at least partially inserted into the patient 130. The instrument may for example be a medical device or medical implant that is controlled or deployed by an instrument subsystem 142. The instrument 140 has a proximal portion 144 and a distal portion 146. In some circumstances, the instrument 140 can be completely inside patient (e.g., both the proximal portion 144 and the distal portion 146 located inside the patient), as may be the case for implants such as tissue anchors or clips. In other circumstances, the instrument 140 can be partially inside the patient, (e.g., only the distal portion is inside the patient), as may be the case for catheters, guidewires, probes, and other devices that are removed from the patient at the end of procedure. A tissue engagement sensor 150 is coupled to the distal end 146 of the instrument 140, to measure engagement of the instrument 140 with tissue 160 inside the patient 130. The tissue engagement sensor 150 may for example be glued to the distal end 146 by an adhesive, or may be held in place by a sleeve, slot, screw, pin, or other fixture, or may be fabricated as part of the distal end 146.

The tissue engagement sensing system 100 also includes a magnetic field subsystem 165, which includes a magnetic field generator 170 and a magnetic field sensor 180. In some embodiments, the magnetic field generator 170 and magnetic field sensor 180 may be the same device. Example magnetic field generators 170 and magnetic field sensors 180 may include devices manufactured by Northern Digital Inc., Polhemus, Radwave, and FreeNav. In some embodiments, the magnetic field subsystem may also include a processor circuit 162, display 164, and/or input device 166.

The magnetic field subsystem 165 may for example be used to excite vibrations in the tissue engagement sensor, which are detected by the ultrasound probe 110. As described above, the strength or frequency of the vibration signal may correspond to or be proportional to the amount of tissue covering the tissue engagement sensor 150, so that the strength or frequency of the vibration signal is a corresponding or proportional indicator on the extent to with the distal portion 146 of the instrument 140 is engaged with the tissue 160. Depending on the implementation, the magnetic field subsystem 165, ultrasound console 120, and/or instrument subsystem 142 can be in communication with one another, or one or more of these systems may share a processor circuit, display, input device, or other components.

Before continuing, it should be noted that the examples described above are provided for purposes of illustration, and are not intended to be limiting. Other devices and/or device configurations may be utilized to carry out the operations described herein.

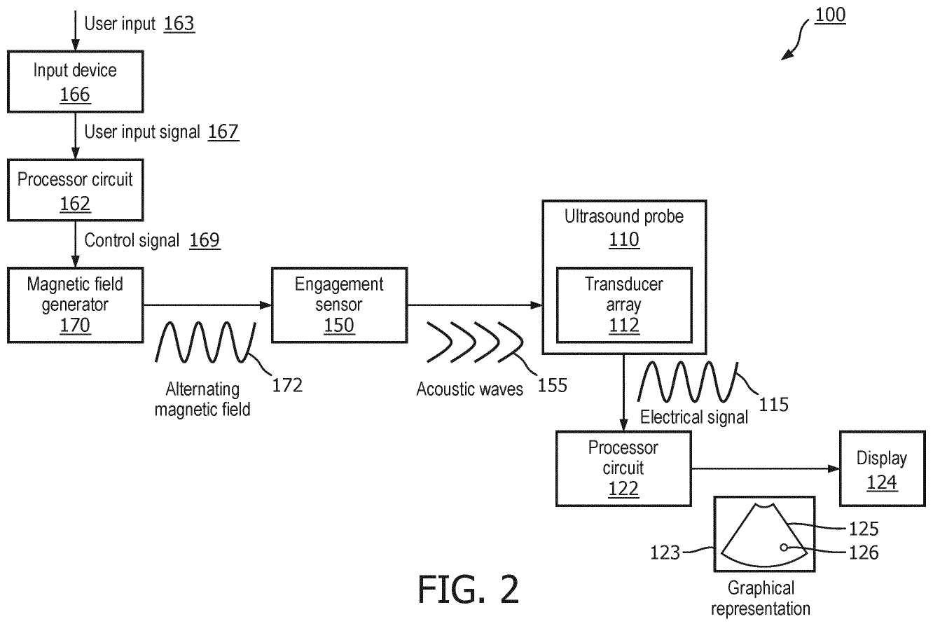

FIG. 2 is a schematic representation, in block diagram form, of at least a portion of an example tissue engagement sensing system 100, in accordance with at least one embodiment of the present disclosure. In the example shown in FIG. 2, the tissue engagement sensing system 100 includes an input device 166 that accepts a user input 163 and generates a user input signal 167 in response. The user input signal 167 is received by the processor circuit 162, which generates a control signal 169 for the magnetic field generator 170. Under the influence of the control signal 169, the magnetic field generator 170 produces an alternating magnetic field 172, which oscillates at a frequency f₁.

The MEMS tissue engagement sensor 150 experiences the alternating magnetic field 172, which may for example be a regular, controlled oscillation, with a constant amplitude and frequency. The alternating magnetic field 172 causes a magneto-restrictive material to vibrate (as described below) at a fundamental frequency f₂, which may for example be a multiple of the magnetic field frequency f₁ (e.g., a first vibration mode f₂=f₁, a second vibration mode f₂=2f₁, a third vibration mode f₂=3f₁, etc.). If the tissue engagement sensor 150 is immersed in a fluid such as blood, the vibration of the tissue engagement sensor 150 generates acoustic waves 155 in the fluid, with a frequency f₂.

The transducer array 112 (in receive mode) of the ultrasound probe 110 receives the acoustic waves 155 and generates electrical signals 115 representative of received acoustic waves 155. The transducer array 112 may for example have a center frequency f₃, a minimum detectable frequency fₘᵢₙ and a maximum detectable frequency fₘₐₓ. It may therefore be desirable to design the engagement sensor 150 such that f₂ falls between fₘᵢₙ and fₘₐₓ, and preferably close to f₃, in order for the ultrasound probe 110 to detect the acoustic waves 155. The electrical signals 115 are received by the processor circuit 122, where they are processed into an image or graphical representation 123 and shown on the display 124.

The graphical representation 123 may be an ultrasound image that depicts the tissue engagement sensor 150 at its given location within the anatomy. In an example, the graphical representation 123 may depict a field of view 125 of the transducer array 112 that is darker (e.g., black/darker grey) and a lighter region 126 (e.g., white/lighter grey) at a location within the field of view that is representative of the tissue engagement sensor 150. The location of lighter region 126 within the field of view 125 depicted in the graphical representation 123 corresponds to the location within the anatomy that the tissue engagement sensor 150 is located. In some instances, the ultrasound image depicts only the tissue engagement sensor 150 at its given location. In other instances, the ultrasound image depicts the tissue engagement sensor 150 at its given location as well as other structures (e.g., tissue/anatomy, an instrument that the tissue engagement sensor 150 is coupled to, etc.) within the field of view 125. In other examples, the graphical representation may be an intensity graph, dial, or other indicator that isolates the characteristic frequency f₂ of the engagement sensor 150 (e.g., that filters out all incoming frequencies except f₂).

The amplitude, acoustic pressure, or acoustic intensity of the acoustic waves 155 at any given time depends on the number of teeth or comb teeth of the engagement 150 that are currently vibrating. Similarly, the amplitude of the electrical signals 115 corresponds to the acoustic pressure and/or acoustic intensity of the acoustic waves 155. Thus, the brightness of the lighter region 126 (representative of the tissue engagement sensor 150) on the display 124, or the magnitude of a value shown graphically in the graphical representation 123, corresponds to or is proportional to the number of teeth or teeth that are currently obstructed by the tissue. Thus, these values can be used as an accurate measure of tissue engagement by the tissue engagement sensor 150, as can the frequency of oscillation.

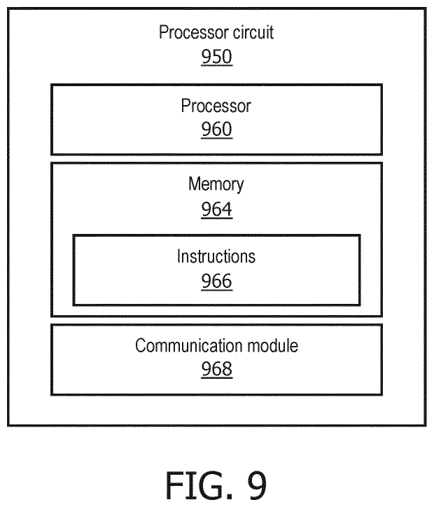

In some instances, the measure of tissue engagement can be a distance that the engagement sensor extends into tissue. The measure of tissue engagement can have a continuous scale (e.g., in percent, microns, millimeters, or other appropriate unit). In other instances, the measure of tissue engagement can have a finite scale. For example, the finite scale could include three values (no engagement-none of the teeth contact tissue, partial engagement-some of the teeth contact tissue and some of the teeth do not contact tissue, full engagement-all of the teeth contact tissue) or other suitable quantity of values. In some instances, the amplitude of the electrical signals 115 and/or the brightness of the lighter region 126 is stored and associated in a memory (e.g., memory 964 of FIG. 9) with a corresponding measure of tissue engagement (e.g., a look-up table with amplitude/brightness values and corresponding distances). In some aspects, the emitted frequency may be used instead or in addition.

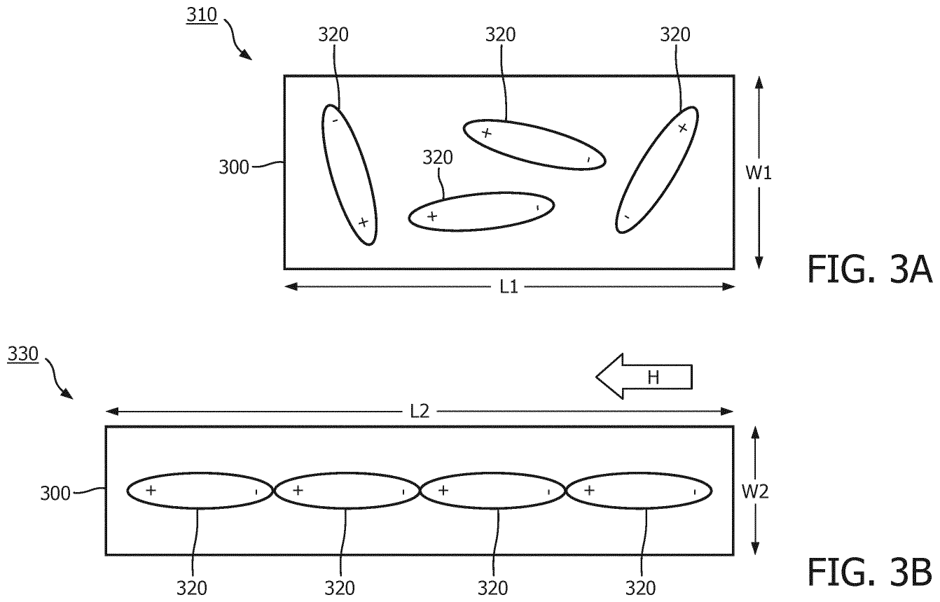

FIG. 3A is a schematic representation of an exemplary magneto-elastic member 300, in accordance with at least one embodiment of the present disclosure. The magneto-elastic member 300 comprises a magneto-elastic material such as terfenol-D, permendur, nickel, and/or alloys of nickel, cobalt, iron, and/or aluminum (e.g., cobalt and nickel, aluminum and iron (lu14 Alfer), Co—Ni, Ni—Co ferrite). In the example shown in FIG. 3A, the magneto-elastic member 300 is in a relaxed state 310, wherein magnetic dipoles 320 within the magneto-elastic member 300 are randomly or quasi-randomly oriented. In the relaxed state 310, the magneto-elastic member 300 has a length L1 and a width W1.

FIG. 3B is a schematic representation of the exemplary magneto-elastic member 300 of FIG. 3A, in accordance with at least one embodiment of the present disclosure. In the example shown in FIG. 3B, the magneto-elastic member 300 is acted on by a magnetic field H, such that the magnetic dipoles 320 within the magneto-elastic member 300 align or partially align with the magnetic field H. This results in an elongated state 330, wherein, the magneto-elastic member 300 has a length L2 that is greater than the length L1 of FIG. 3A, and a width W2 that is narrower than the width W1 of FIG. 3A. In an oscillating magnetic field, the magneto-elastic member 300 can oscillate between the relaxed state 310 and the elongated state 330.

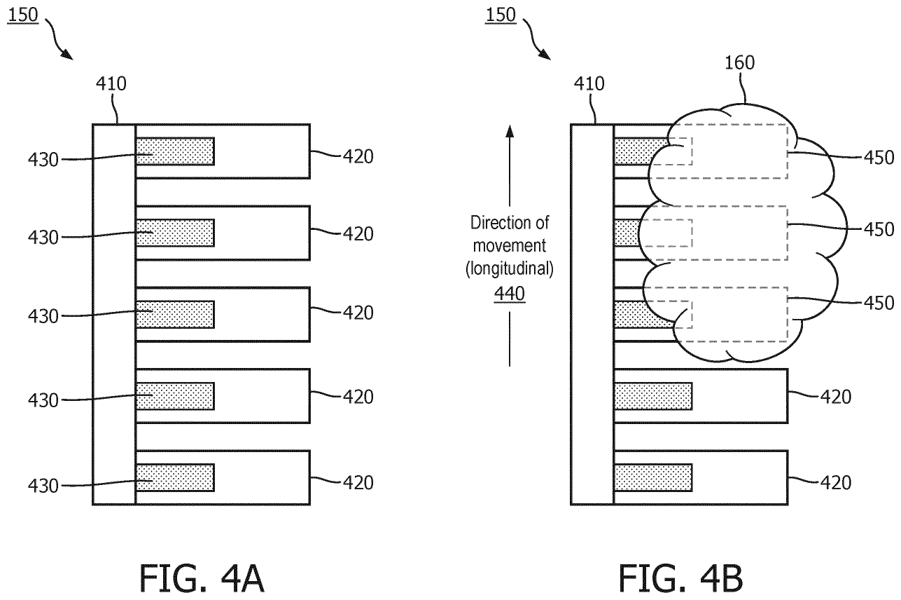

FIG. 4A is a schematic, diagrammatic bottom view of an exemplary tissue engagement sensor 150, in accordance with at least one embodiment of the present disclosure. In the example shown in FIG. 4A, the tissue engagement sensor 150 includes a longitudinal brace or support member 410, from which a plurality of laterally oriented teeth 420 project. The laterally oriented teeth 420 extend perpendicular or orthogonal to the longitudinal brace or support member 410 in FIG. 4A. In other examples, the laterally oriented teeth 420 extend at an oblique or non-perpendicular/non-orthogonal angle to the longitudinal brace or support member 410 in FIG. 4A. The brace or support member 410 and the teeth 420 may be made of the same material or different materials, and may for example be fabricated using semiconductor manufacturing techniques common to microelectromechanical systems (MEMS). As such, either or both of the support member 410 and the teeth 420 may be made of any combination of metals, semiconductors, oxides, or polymers. In the example shown in FIG. 4A, the teeth 420 are cantilevered, e.g., fixed or supported only at one end. However, in other embodiments there may be a second support member 410 (whether connected to the first support member 410 or otherwise), such that the teeth 420 are fixed or supported at both ends. In such cases, the oscillation of the teeth 420 may for example occur at the center of the tooth, which is free to oscillate up and down while the ends are anchored, much like a guitar string.

Each tooth 420 includes a magneto-elastic member 430. In the example shown in FIG. 4A, the magneto-elastic members 430 also project from the longitudinal brace or support member 410, and extend partway down the length of the teeth 420. In other embodiments, the magneto-elastic members 430 may or may not be attached to the brace or support member 410, and can extend for any portion of the length of the teeth 420, including in some embodiments the entire length. In other embodiments, the teeth 420 and magneto-elastic members 430 may be the same objects, e.g., the teeth 420 may include or be made of a magneto-elastic material. Furthermore, although the magneto-elastic member 430 is shown here at the bottom of the tooth 420, it can, instead or in addition, be placed on the side or top of the tooth 420, and/or at any position along the length of the tooth 420 that is capable of causing the tooth 420 to oscillate. In the example shown in FIG. 4A, each tooth 420 is cantilevered, having one side anchored to the support member 410 and one side unattached and thus free to oscillate.

As described above, the magneto-elastic material of the magneto-elastic members 430 elongates or contracts in response to magnetic fields. Thus, an oscillating magnetic field can cause the magneto-elastic members 430 to increase and decrease in length in a periodic manner, leading to mechanical oscillation (e.g., in and out of the page) of the teeth 420. Thus mechanical oscillation then produces acoustic waves that can be detected and measured by an ultrasound system.

The addition of a second, third, etc. tooth or magneto-elastically driven oscillator changes the generated acoustic waves relative to when only a single tooth or magneto-elastically driven oscillator generates the acoustic waves. For example, the change can be to a quantity of acoustic waves, an acoustic pressure, an acoustic intensity, an amplitude, and/or a frequency, etc., as described below. The change can be an increase (add to) or a decrease (subtract from). For example, the acoustic waves generated by the second tooth or magneto-elastically drive oscillator can add to the amplitude of the acoustic waves generated by the first tooth or magneto-elastically driven oscillator.

FIG. 4B is a schematic, diagrammatic bottom view of an exemplary tissue engagement sensor 150, in accordance with at least one embodiment of the present disclosure. In the example shown in FIG. 4B, the tissue engagement sensor 150 is moving in a longitudinal direction 440 toward the tissue 160. In other examples, the movement of the tissue engagement sensor 150 relative to the tissue 160 (e.g., to contact the tissue) can be any type of movement in any direction, such as a rotational, longitudinal, forward, backward, left, right, etc., and/or any combination). As the tissue engagement sensor 150 comes into contact with the tissue 160, some of the teeth are covered by the tissue 160. The vibration of these covered teeth 450 is damped or suppressed, whereas the uncovered teeth 420 can continue to oscillate normally. Thus, under the influence of an oscillating magnetic field, the strength, magnitude, amplitude, or frequency of the acoustic signal generated by the tissue engagement sensor 150 may correspond to or be proportional to the number of teeth that are covered.

In the example of FIG. 4B, three teeth 450 are covered by the tissue 160, while two teeth 420 remain uncovered, so that the acoustic signal produced by the tissue engagement sensor 150 may be approximately two-fifths or 40% as strong as the unencumbered tissue engagement sensor 150 if FIG. 4A. In this example, the tissue engagement sensor 150 may be affixed to a medical implant such as a tissue anchor, at a location such that the engagement of exactly three teeth (e.g., a signal strength reduction of approximately 60%) indicates that the tissue anchor has been driven into the tissue to a desired depth, whereas the engagement of only one or two teeth (e.g., a signal strength reduction of 20-40%) would indicate insufficient depth, and engagement of four or five teeth (e.g., a signal strength reduction of 80-100%) would indicate excessive depth. Thus, when attached to a medical instrument, the tissue engagement sensor 150 can, passively and wirelessly, provide an accurate measure of how much tissue the medical instrument has engaged.

Depending on the implementation, the teeth may have a width and/or thickness of 10-100 microns and a length of 100-1000 microns, and may be spaced 10-100 microns apart along the brace or support member 410, although other dimensions both larger and smaller may be used instead or in addition. Similarly, the tissue engagement sensor 150 may include only a single tooth 420, thus providing a binary (yes/no) indication of tissue engagement, or may include dozens or even hundreds of teeth 420, thus providing a more precise indication of tissue engagement. For example, with a tissue engagement sensor 150 that included 100 teeth 420, the percent reduction in signal strength could be approximately equal to the percent of tissue engagement by the tissue engagement sensor 150.

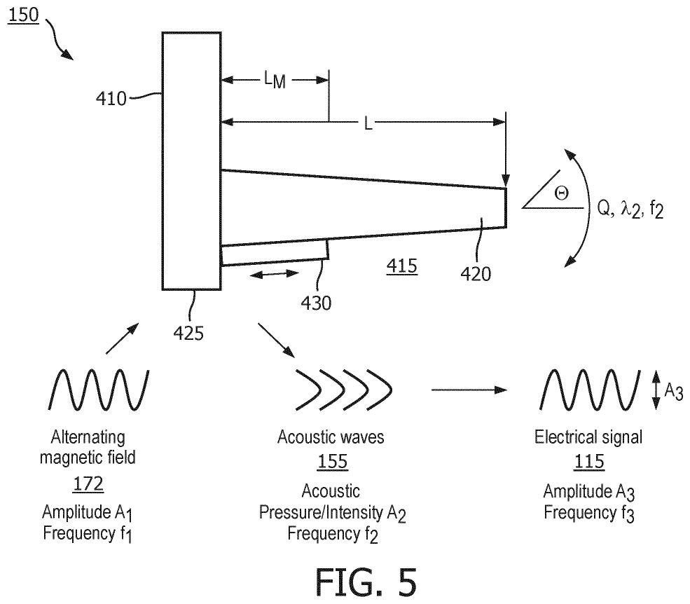

FIG. 5 is a schematic, diagrammatic side view of an exemplary tissue engagement sensor 150, in accordance with at least one embodiment of the present disclosure. Visible are the brace or support member 410, a tooth 420, and a magneto-elastic member 430. A surface 425 of the support member 410 can be coupled to an outer surface of the instrument 140 to couple the engagement sensor 150 and the instrument 140. Each tooth 420 and magneto-elastic member 430 form a respective magneto-elastically driven oscillator. The tooth 420 is positioned within a well, cavity, or clearance region 415 that provides room for it to vibrate. As described above, an alternating magnetic field 172 (also referred to as an oscillating magnetic field) of amplitude A₁ and frequency f₁ causes the magneto-elastic member to expand and contract, such that its length Lₘ increases and decreases in a periodic manner. This causes the tooth 420, with a length of L, to vibrate up and down through an angle ⋅ and an amplitude Q that may be functions of the intensity/amplitude of the magnetic field 172 and the length L of the tooth 420. The vibration also has a characteristic wavelength λ₂ and characteristic frequency f₂ that may be functions of the length L of the tooth 420. In some embodiments, the frequency f₁ of the alternating magnetic field 172 may be selected to be a whole fraction (e.g., ½, ¼, etc.) of the frequency f₂, in order to maximize the resonance of the tooth and thus strengthen the output signal. It is understood that the tooth 420 has a certain degree of flexibility that allows it to oscillate through the angle Θ; depending on the implementation, a stiffer material may oscillate through a smaller angle and a more flexible material may oscillate through a larger angle.

Vibration of the tooth 420 produces acoustic waves 155 with a frequency f₂ (e.g., equal to and/or corresponding to the characteristic frequency f₂ of the vibration of the tooth) and an amplitude (e.g., acoustic pressure and/or acoustic intensity) of A₂, which corresponds to or is proportional to amplitude Q and the number of teeth 420 that are unobstructed and free to vibrate. These acoustic waves 155 is then received by an ultrasound transducer and turned into an electrical signal 115 of frequency f₃ and amplitude A₃. The values of f₃ and/or A₃ can then be used as an accurate measure of the tissue engagement of the tissue engagement sensor 150. Each tooth 420 and magneto-elastic member 430 together form a respective magneto-elastically driven oscillator that is capable of vibrating independently of the other teeth in the tissue engagement sensor 150. The tissue engagement sensor 150 can include one or a plurality of magneto-elastically driven oscillators.

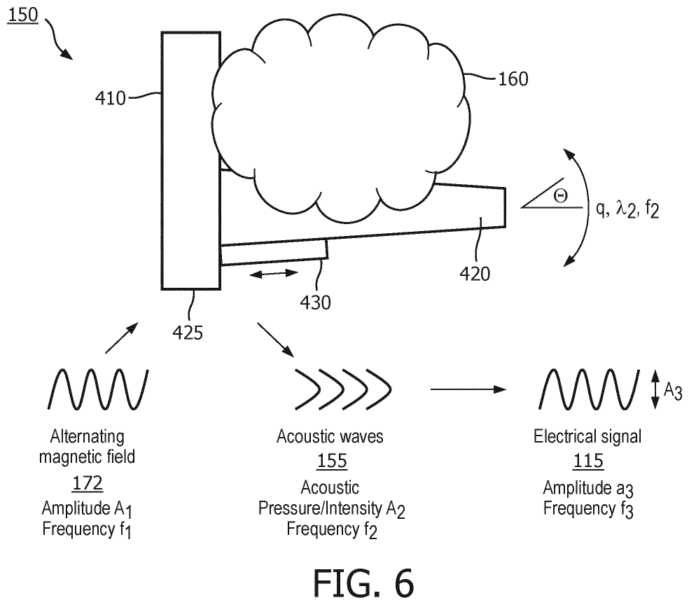

FIG. 6 is a schematic, diagrammatic side view of an exemplary tissue engagement sensor 150, in accordance with at least one embodiment of the present disclosure. Visible are the brace or support member 410, a tooth 420, and a magneto-elastic member 430. In the example shown in FIG. 6, the tooth 420 is in contact with tissue 160. In the presence of the alternating magnetic field 172 (as in FIG. 5), contact between the tooth 420 and the tissue 160 limits vibration of the tooth to an angle. (which may be smaller than the angle. of FIG. 5), a wavelength 22, a frequency f₂, and an amplitude of q (which may be smaller than the amplitude Q of FIG. 5, and may in some cases be negligible). In some aspects, the frequency f₂ of FIG. 6 can be different than the frequency f₂ of FIG. 5. The frequency f₂ of FIG. 6 can be larger or smaller than the frequency f₂ of FIG. 5. In some aspects, the wavelength λ₂ of FIG. 6 can be different than the wavelength λ₂ of FIG. 5. The wavelength λ₂ of FIG. 6 can be larger or smaller than the wavelength λ₂ of FIG. 5.

As a result of the change in vibration of the tooth 420, the acoustic waves 155 emitted by the tooth 420 have a frequency f₂, as well as an amplitude a₂, which may smaller than the amplitude A₂ of FIG. 5, and may in some cases be negligible. Consequently, the ultrasound system produces an electrical signal 115 of frequency f₃ and amplitude a₃ (which may be less than the amplitude A₃ of FIG. 5, and may in some cases be negligible). In some aspects, the frequency f₃ of FIG. 6 can be different than the frequency f₃ of FIG. 5. The frequency f₃ of FIG. 6 can be larger or smaller than the frequency f₃ of FIG. 5.

In some aspects, at least one of the frequency or the amplitude of the vibration of the tooth 420, the acoustic waves 155, and/or the electrical signal 115 of FIG. 6 is different than the frequency and the amplitude of the vibration of the tooth 420, the acoustic waves 155, and/or the electrical signal 115 of FIG. 5. In some aspects, both the frequency and the amplitude are different. For example, for given energy input (e.g., amplitude A1 and frequency f₁ of the alternating magnetic field 172), the frequency can be inversely proportional to the amplitude. For example, if the frequency decreases from FIG. 5 to FIG. 6, then the amplitude increases from FIG. 5 to FIG. 6 (and vice versa). In some aspects, the frequency is the same and the amplitude is different. In some aspects, the frequency is different and the amplitude is the same.

Each tooth 420 that is obstructed by tissue 160 may contribute less to the overall amplitude of the acoustic signature 115 of the tissue engagement sensor 150 (relative to the if the tooth 420 is unobstructed by the tissue) and thus the amplitude of the electrical signal 115 produced by the ultrasound system. Either the frequency or the amplitude of the electrical signal 115, or combinations thereof, may be used as a measure of tissue engagement.

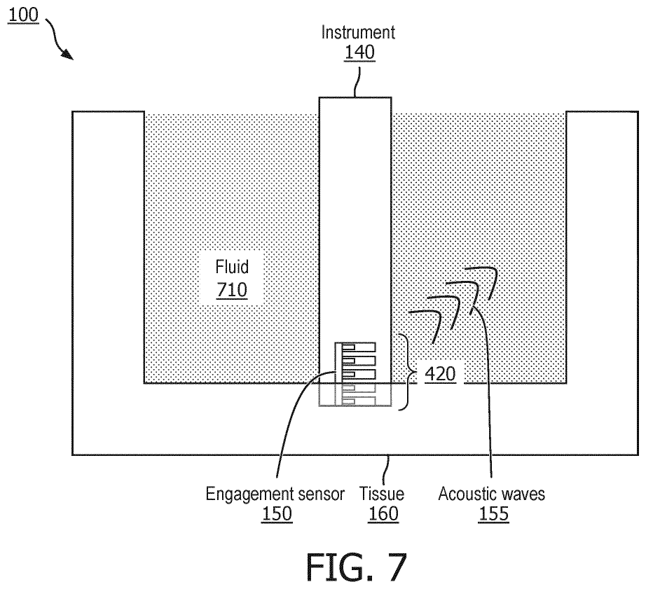

FIG. 7 is a schematic, diagrammatic representation, in block diagram form, of at least a portion of an example tissue engagement sensing system 100, in accordance with at least one embodiment of the present disclosure. In the example shown in FIG. 7, the instrument 140 includes a tissue engagement sensor 150 at its distal end, with two of five teeth 420 engages with tissue 160 and three of five teeth 420 not engaged. This results in a reduction (e.g., a 40% reduction) of the amplitude or intensity of sound waves 155 emitted by the engagement sensor 150 into the surrounding fluid 710 (e.g., blood, saline, etc.). As described above, this reduction in intensity can be detected and measured by the ultrasound system, providing an accurate measurement of how many teeth 420 of the tissue engagement sensor 150 are engaged by the tissue 160, which in turn provides an indication of the distance of engagement of the instrument 140 with the tissue 160. In some aspects, the change in frequency of the sound waves 155 can also be used can be detected and measured by the ultrasound system, providing an accurate measurement of how many teeth 420 of the tissue engagement sensor 150 are engaged by the tissue 160, which in turn provides an indication of the distance of engagement of the instrument 140 with the tissue 160. For example, the amplitude/intensity and/or frequency of sound waves 155 emitted by the engagement sensor 150 into the surrounding fluid 710 (e.g., blood, saline, etc.) without any engagement of the sensor 150 into tissue can be pre-determined and/or stored by the processing circuit. Or the amplitude/intensity and/or frequency of sound waves 155 emitted by the engagement sensor 150 into the surrounding fluid 710 (e.g., blood, saline, etc.) without any engagement of the sensor 150 into tissue can be determined by the processing circuit during the medical procedure itself (e.g., before engagement of the sensor 150 into tissue). The processing circuit can compare the amplitude/intensity and/or frequency of the sound waves 155 without engagement of the sensor 150 into tissue and the amplitude/intensity and/or frequency of the sound waves 155 with engagement of the sensor 150 into tissue (how much difference in amplitude/intensity, how much difference in frequency), to determine the degree of engagement of the sensor 150 with the tissue. For example, the relationship between the difference in amplitude/intensity and/or difference in frequency with distance of the sensor 150 into the tissue can be stored by the processing circuit (e.g., a look up table). The instrument 140 may for example be a clip used for grasping of heart valve leaflets in a regurgitation repair, an anchorage of a chordae repair device in the heart, a needle used for biopsy of tissue, etc., such that the tissue engagement sensor 150 is used to measure engagement of these instruments with the tissue appropriate to the procedure being performed.

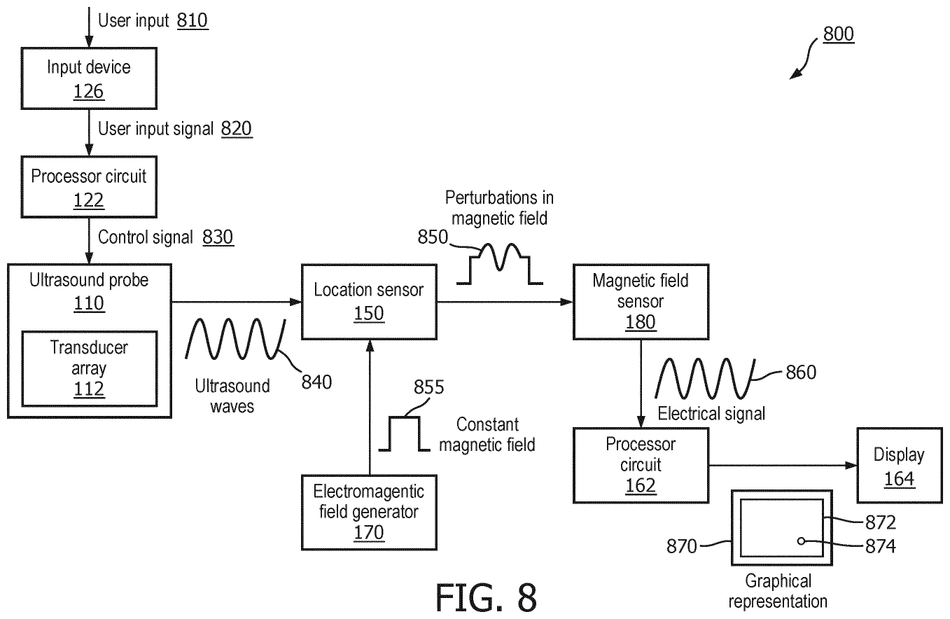

FIG. 8 is a schematic representation, in block diagram form, of at least a portion of an example instrument tracking system 800 employing components of the tissue engagement sensing system 100, in accordance with at least one embodiment of the present disclosure. In the example shown in FIG. 8, the tissue engagement sensing system 100 is operated in reverse (e.g., relative to operation of the system 100 shown in FIG. 2) such that the ultrasound system is used to excite vibrations in the engagement sensor 150, generating an oscillating magnetic field which is detected by the magnetic field sensor 180 for purposes of tracking the position of the engagement sensor 150, and therefore of the instrument to which it is affixed. Thus, it is understood that, depending on the implementation and usage, the sensor 150 may be a tissue engagement sensor 150, a location sensor 150, or both. It should be noted with particular emphasis that the same sensor 150, during the same medical procedure, may be used as both a location sensor as shown in FIG. 8 and a tissue engagement sensor as shown in FIG. 2. The magnetic field sensor 180 may for example be configured to locate a center point or strongest point of the oscillating magnetic field.

In this example, the instrument tracking system 800 includes an input device 126 that accepts a user input 810 and generates a user input signal 820 in response. The user input signal 820 is received by the processor circuit 122, which generates a control signal 830 for the transducer array 112 of the ultrasound probe 110. Under the influence of the control signal 830, the transducer array 112 (in transmit mode) produces ultrasound waves 840, which oscillate at a frequency f₁ and amplitude a₁ through a fluid such as blood.

If immersed in the fluid, the MEMS tissue engagement sensor 150 vibrates in response to the ultrasound waves 840, causing a magneto-restrictive material to vibrate (as described above) at an amplitude a₂ and a fundamental frequency f₂, which may for example be a multiple or fraction of the ultrasound frequency f₁ (e.g., a first vibration mode f₂=f₁, a second vibration mode f₂=2f₁, a third vibration mode f₂=½f₁, etc. Both a₂ and f₂ may be affected by tissue engagement, as described above. The vibration of the tissue engagement sensor 150 excited the magneto-elastic members, which generate an oscillating magnetic field 850, with an amplitude a₃ and a frequency f₃ (which may be equal to f₂). In some embodiments, this may occur under the influence of a constant magnetic field 855, in which case the oscillating magnetic field 850 will show up as perturbations of the constant magnetic field 855. It is noted that the oscillating magnetic field 855 can include any type of oscillation. For example, if the ultrasound energy is delivered with a constant frequency and amplitude, then the oscillating magnetic field 855 may also have a constant frequency and amplitude. However, if the ultrasound energy is delivered in pulses with gaps in between, then the oscillating magnetic field 855 may similarly appear in pulses with gaps in between, and may not be a regular, controlled oscillation with constant amplitude and frequency, but rather be perturbations in the magnetic field. It is thus understood that the oscillation can have a decaying amplitude, a changing frequency, can be irregular or uncontrolled, etc.

The magnetic field sensor 180 receives the oscillating magnetic field (or perturbations of the constant magnetic field) 850, and generates electrical signals 860 representative of the detected oscillating magnetic field 850. The electrical signals 860 are received by the processor circuit 162, where they are processed into an image or graphical representation 870 and shown on the display 124. In an example, the graphical representation may be an image of the tissue engagement sensor 150. The image can depict a portion 872 of the anatomy in which the magnetic field is detectable by the magnetic field sensor 180. The region 874 corresponds to the tissue engagement sensor 150 within the portion 872 of the anatomy. The image may or may not include the instrument to which the tissue engagement sensor 150 is coupled, tissue, and other nearby anatomical structures of the patient. In other examples, the graphical representation 870 may be a numeric, alphanumeric, cartesian, polar, or other representation indicative of a location (e.g., an X-Y, X-Z, Y-Z, or X-Y-Z location) of the engagement sensor 150 relative to the magnetic field sensor 180.

Thus, the same hardware used to measure tissue engagement can also be run in reverse (whether sequentially or in parallel) to track the location of the engagement sensor 150. More generally, it is to be appreciated that the tissue engagement sensor 150 is a passive, wireless device that can be excited by either ultrasound waves or an oscillating magnetic field, and can be detected either by an ultrasound probe in receive mode or by a magnetic field sensor. All such combinations can employ the same or similar hardware, explicitly fall within the scope of the present disclosure.

It is noted that adding a second, third, fourth, etc. tooth or magneto-elastically driven oscillator changes the oscillating magnetic field relative to when only a single tooth or magneto-elastically driven oscillator generates the oscillating magnetic field. For example, the change can be to an amplitude of the oscillations/perturbations 850, a frequency of the oscillations/perturbations 850, the magnetic field strength, magnetic flux density, etc. Similarly, adding a second, third, fourth, etc. tooth or magneto-elastically driven oscillator changes the electrical signal 860 relative to the electrical signal 860 with only a single tooth or magneto-elastically driven oscillator. For example, the change can be to an amplitude of the electrical signal 860, a frequency of the electrical signal 860, etc. The change can be an increase (add to) or a decrease (subtract from). For example, the oscillations/perturbations in the magnetic field generated by the second magneto-elastically drive oscillator can add to the amplitude of the oscillations/perturbations in the magnetic field generated by the first magneto-elastically driven oscillator. For example, the amplitude of the electrical signal 860 with the second magneto-elastically drive oscillator can add to the amplitude of the electrical signal 860 with the first magneto-elastically driven oscillator. The location of the sensor 150 can be determined based on the amplitude, frequency, etc., and/or combinations thereof.

FIG. 9 is a schematic diagram of a processor circuit 950, in accordance with at least one embodiment of the present disclosure. The processor circuit 950 may be implemented in the system 100, the system 800, processor circuits 122 or 162, or other devices or workstations (e.g., third-party workstations, network routers, etc.), or on a cloud processor or other remote processing unit, as necessary to implement the method. As shown, the processor circuit 950 may include a processor 960, a memory 964, and a communication module 968. These elements may be in direct or indirect communication with each other, for example via one or more buses.

The processor 960 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 960 may also comprise another hardware device, a firmware device, or any combination thereof configured to perform the operations described herein. The processor 960 may also be implemented as a combination of computing devices, e.g., a combination of a DSP and a microprocessor, a plurality of microprocessors, one or more microprocessors in conjunction with a DSP core, or any other such configuration.

The memory 964 may include a cache memory (e.g., a cache memory of the processor 960), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 964 includes a non-transitory computer-readable medium. The memory 964 may store instructions 966. The instructions 966 may include instructions that, when executed by the processor 960, cause the processor 960 to perform the operations described herein. Instructions 966 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 968 can include any electronic circuitry and/or logic circuitry to facilitate direct or indirect communication of data between the processor circuit 950, and other processors or devices. In that regard, the communication module 968 can be an input/output (I/O) device. In some instances, the communication module 968 facilitates direct or indirect communication between various elements of the processor circuit 950 and/or the system 100 or the system 800. The communication module 968 may communicate within the processor circuit 950 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the ultrasound probe 110 or magnetic field sensor 180) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

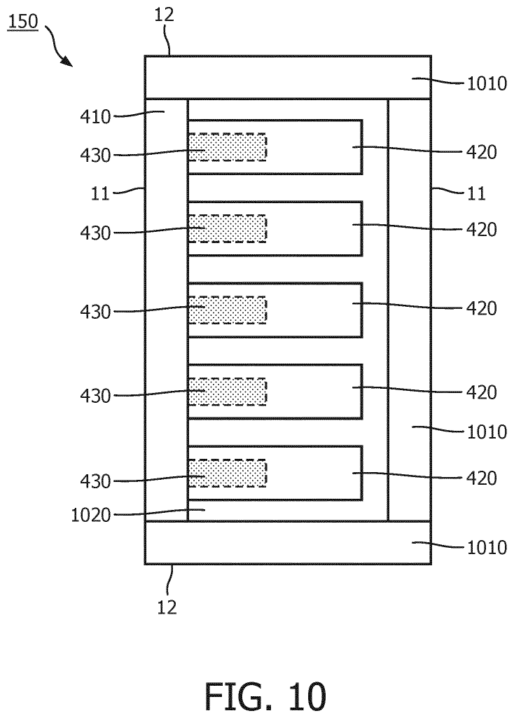

FIG. 10 is a schematic, diagrammatic top view of an exemplary tissue engagement sensor 150, in accordance with at least one embodiment of the present disclosure. Visible are the support member 410, teeth 420, and magneto-elastic members 430. In the example shown in FIG. 10, the tissue engagement sensor 150 also includes sidewalls 1010 and a bottom 1020.

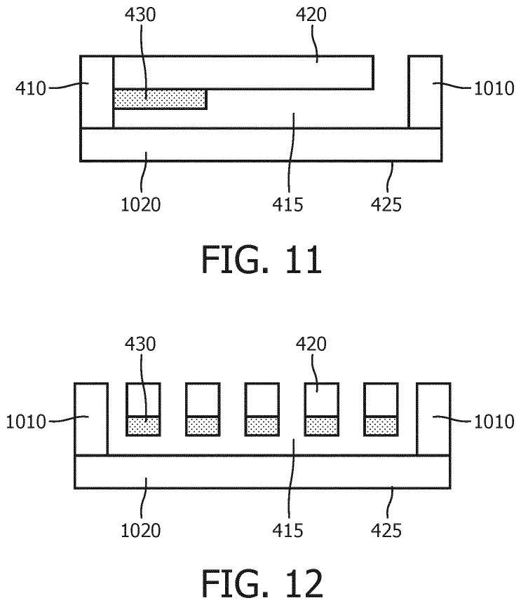

FIG. 11 is a schematic, diagrammatic side cross-sectional view along section line 11 -11 in FIG. 10 of the exemplary tissue engagement sensor 150, in accordance with at least one embodiment of the present disclosure. Visible are the support member 410, a tooth 420, a magneto-elastic member 430, a sidewall 1010, the well, cavity, or clearance region 415 (providing room for the tooth 420 to vibrate), and the bottom 1020. In the example shown in FIG. 10, the tissue engagement sensor 150 also includes sidewalls 1010 and a bottom 1020. The bottom 1020 (e.g., the surface 415) may for example be coupled to the outer surface of a medical instrument to couple the tissue engagement sensor 150 to the medical instrument. The sidewalls 1010 and the bottom 1020 can at least partially define the well, cavity, or clearance region 415. The sidewalls 1010 and the bottom 1020 may advantageously allow the teeth 420 to vibrate (within the well, cavity, or clearance region 415) even when the surface 425 is coupled to the outer surface of the medical instrument.

FIG. 12 is a schematic, diagrammatic front cross-sectional view along section line 12-12 in FIG. 10 of the exemplary tissue engagement sensor 150, in accordance with at least one embodiment of the present disclosure. Visible are the teeth 420, magneto-elastic members 430, two sidewalls 1010, the well, cavity, or clearance region 415 (providing room for the teeth 420 to vibrate), and the bottom 1020 (including, e.g., the surface 425).

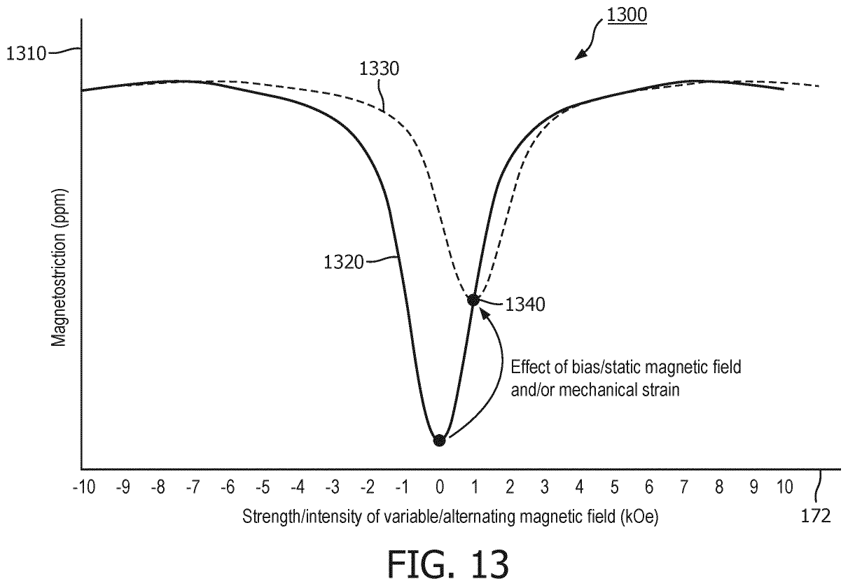

FIG. 13 is a graph 1300 showing magnetostriction or magneto-strictive response 1310 of a magneto-elastic material as a function of the strength of the variable (alternating) magnetic field 172 in kiloOersteds (kOe), in accordance with at least one embodiment of the present disclosure. A first curve 1320 shows the magneto-strictive response of the material when no constant/fixed/bias magnetic field or no mechanical strain is applied to the material. In this case, the magnetostriction 1310 of the material is approximately zero when the strength of the alternating magnetic field 172 is equal to zero, and rises to a nonzero value 1335 (e.g., nonzero elongation of the material) at field strengths greater or less than zero.

A second curve 1330 shows the effect of applying a constant magnetic field in addition to the alternating magnetic field 172, and/or of applying a mechanical strain to the magneto-elastic material. The constant magnetic field can also be referred to as a fixed magnetic field or biased magnetic field. The mechanical strain can be applied as a result of contact with the magneto-elastic material. In this case, the magneto-strictive response is altered, such that when the strength of the alternating magnetic field 172 is equal to zero, the magnetostriction 1310 of the material is not zero. Rather, the curve 1330 shows a minimum 1340 at a nonzero value of the alternating magnetic field 172.

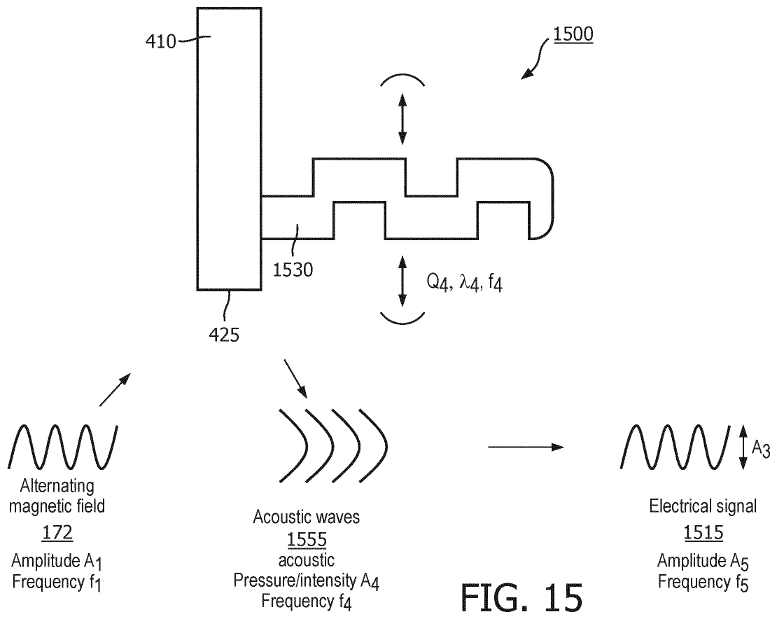

Notably, the area under the second curve 1330 is greater than the area under the first curve 1320. Thus, a fixed or biased magnetic field, or a mechanical strain, can be used to increase the magneto-strictive response 1310 of the magneto-elastic material. It is noted that mechanical strain may be applied more easily to the magneto-elastic material if the entire tooth is made from the magneto-elastic material, and more easily still if the tooth and magneto-elastic member replaced by a magneto-strictive spring, as described below in FIGS. 15-17A.

Overall, the curve 1330 has a larger magneto-strictive response for all values of the alternating magnetic field 172 (zero and non-zero), as a result of the constant magnetic field 172. This larger magneto-strictive response can advantageously be utilized for the sensors (e.g., tissue engagement sensors, location sensors, etc.) described herein.

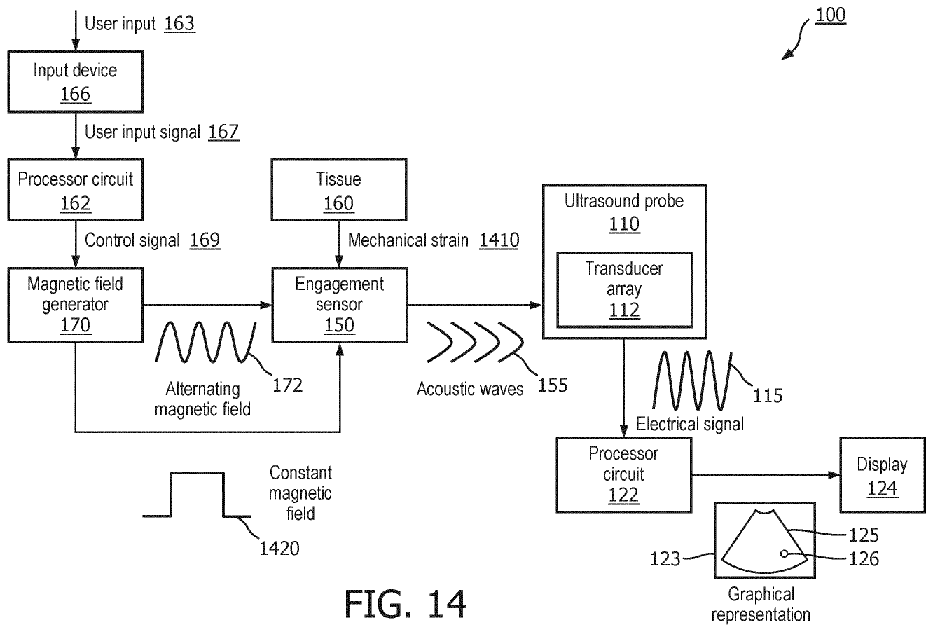

FIG. 14 is a schematic representation, in block diagram form, of an example tissue engagement sensing system 1400, in accordance with at least one embodiment of the present disclosure. The tissue engagement system 1400 is similar to the tissue engagement system 100 of FIG. 2, but with the addition of a mechanical strain 1410 (e.g., applied to the magneto-elastic material by contact with the tissue 160) and/or a constant magnetic field 1420. Because the mechanical strain 1410 and/or constant magnetic field 1420 increase the magneto-strictive response of the tissue engagement sensor 150, the tissue engagement sensor 150 produces acoustic waves 155 of a greater acoustic pressure or intensity relative to FIG. 2, and thus the transducer array 112 produces an electrical signal 115 of greater amplitude relative to FIG. 2.

FIG. 15 is a schematic, diagrammatic side view of an exemplary tissue engagement sensor 1500, in accordance with at least one embodiment of the present disclosure. The tissue engagement sensor 1500 is similar to the tissue engagement sensor 150 of FIG. 5, except that the tooth 420 and magneto-elastic member 430 have been replaced with a magneto-elastic spring 1530 that is attached to the brace or support member 410. The spring 1530 may for example be fabricated from magneto-elastic material using microelectromechanical systems (MEMS) techniques, and may take the form of a 3D coil, a 2D zig-zag, or other shape that is capable of serving the same or a similar function. In some instances, in order to increase the magneto-strictive response of the tissue engagement sensor 1500, the spring 1530 may be made entirely from the magneto-elastic material and includes no additional non-magneto-elastic material.

When no constant/fixed/bias magnetic field or mechanical strain is applied to the spring 1530, then in response to the alternating magnetic field 172 of amplitude A₁ and frequency f₁, the spring 1530 oscillates with an amplitude Q₄, wavelength λ₄, and frequency f₄. This produces acoustic waves 1555 with an acoustic pressure or intensity A₄ and frequency f₄, causing the ultrasound transducer array to produce an electrical signal 1515 of amplitude A₅ and frequency f₅.

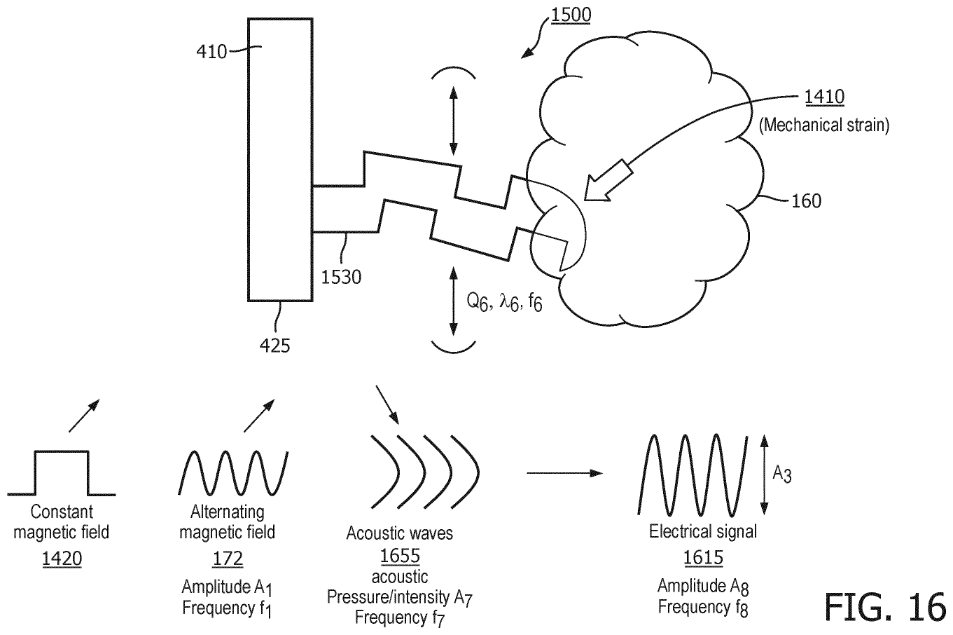

FIG. 16 is a is a schematic, diagrammatic side view of the exemplary tissue engagement sensor 1500 of FIG. 15, loaded with a mechanical strain 1410 and/or a constant/fixed/bias magnetic field 1420, in accordance with at least one aspect of the present disclosure. The constant magnetic field 1420 may for example be applied by a fixed magnet or electromagnet, or by the same magnetic field generator that produces the alternating magnetic field 172. The mechanical strain may for example be produced by contact between the tissue 160 and the spring 1530. The contact between the tissue 160 and the spring 1530 can be contact that urges the spring 1530 against the tissue 160. This may be similar to FIG. 7, where the engagement sensor is part of an instrument that is being pushed into and/otherwise engages the tissue. The constant magnetic field 1420 and/or mechanical strain 1410 increases the magneto-strictive response of the material comprising the magneto-elastic spring 1530 versus what is shown in FIG. 15. Thus, in response to the alternating magnetic field 172 of amplitude A₁ and frequency f₁, the spring 1530 oscillates with an amplitude Q₆, wavelength λ₆, and frequency f₆, where Q₆ is larger than the amplitude Q₄ of FIG. 15. This produces acoustic waves 1555 with an acoustic pressure or intensity A₇ and frequency f₇, causing the ultrasound transducer array to produce an electrical signal 1515 of amplitude A₈ and frequency f₈, where A₇ and A₈ are larger than the amplitudes A₄ and A₈ of FIG. 15. In some aspects, the frequency f₆, f₇, and/or f₈ (FIG. 16) can be smaller when the spring 1530 experiences mechanical strain, compared to the frequency f₄, f₅ (FIG. 15) when the spring 1530 does not experience mechanical strain.

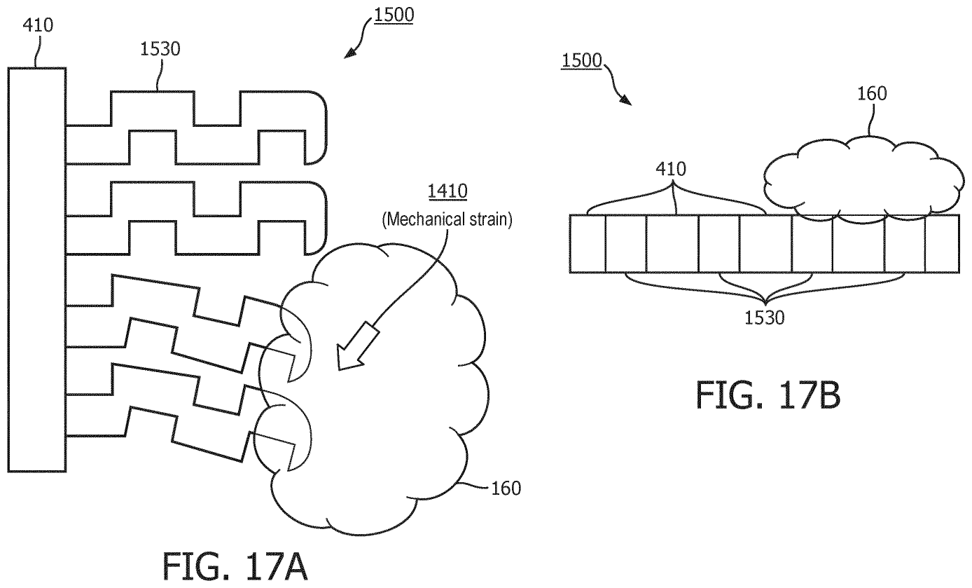

FIG. 17A is a schematic, diagrammatic bottom view of an exemplary tissue engagement sensor 1500, in accordance with at least one embodiment of the present disclosure. The tissue engagement sensor 1500 is similar to the tissue engagement sensor 150 shown in FIG. 4B, except that the magneto-elastic members 430 and teeth 420 have been replaced by magneto-elastic springs 1530 attached to the support member 410. In the example shown in FIG. 17A, two of the springs 1530 are contacted by the tissue 160 in a way that imparts a mechanical strain 1410, while two of the springs 1530 are not engaged by the tissue 160. In this configuration, the springs 1530 that experience the mechanical strain 1410 from the tissue 160 will vibrate with a larger amplitude and thus contribute a stronger acoustic signal, whereas the springs 1530 that do not experience the mechanical strain 1410 will vibrate with a smaller amplitude and thus contribute a weaker acoustic signal. Thus, the strength of the acoustic signal (e.g., the amplitude or intensity) of the sound waves produced by the tissue engagement sensor 1500 increases with the number of springs 1530 that are engaged by the tissue 160.

FIG. 17B is a schematic, diagrammatic front view of the exemplary tissue engagement sensor 1500 of FIG. 17A, in accordance with at least one embodiment of the present disclosure. Visible are the support member 410 and springs 1530. In the example shown in FIG. 17B, two of the springs 1530 are not engaged by the tissue 160, whereas two of the springs 1530 are engaged by the tissue 160 in a way (e.g., on a top surface vs. against the end as in FIG. 17A) that does not impart a mechanical strain, but rather inhibits vibration of the springs 1530. In this configuration, the springs 1530 that are not engaged by the tissue 160 will vibrate with a larger amplitude and thus contribute a stronger acoustic signal, whereas the springs 1530 that engaged by the tissue 160 will vibrate with a smaller amplitude and thus contribute a weaker acoustic signal. Thus, the strength of the acoustic signal (e.g., the amplitude or intensity) of the sound waves produced by the tissue engagement sensor 1500 decreases with the number of springs 1530 that are engaged by the tissue 160.

Whereas the sensor in, e.g., FIGS. 4A-6 may show a reduced acoustic pressure or intensity in proportion to the number of teeth 420 that are engaged by the tissue 160 (see FIG. 4B), the sensor shown in FIGS. 15, 16, and 17A (contact with tissue causes mechanical strain on springs 1530) may show in increased acoustic pressure or intensity in proportion to the number of springs 1530 that are engaged by the tissue 160. FIG. 17B (contact with tissue does not cause mechanical strain on springs 1530) is similar to FIGS. 4A-6, and may show a reduced acoustic pressure or intensity in proportion to the number of springs 1530 that are engaged by the tissue 160.

In the case of FIGS. 4A-6 and 17B, the output signal strength (e.g., acoustic pressure/intensity of the acoustic waves and/or amplitude of the electrical signal) is proportional to the number of teeth/springs that are not in contact with the tissue. Contact with the tissue can decrease the output signal strength. For example, the processor can determine that the teeth/springs that are not in contact with the tissue are contributing most to the acoustic intensity and/or electrical signal and use the quantity of teeth/springs that are not in contact as a measure of how far the sensor extends into tissue. The processor and/or processor circuit can determine that the extent of tissue contact based on the number of teeth/spring not in contact with tissue because the output signal strength decreases with increasing tissue contact. For example, the sensor's total number of teeth/springs, the total length, and/or the output signal strength (e.g., in laboratory, testing, and/or experimental conditions) with none, all, or a portion of the teeth/springs in contact with tissue can be stored in a memory of the processor and/or processor circuit. Depending on the in situ (during the actual procedure) output signal strength, the processor circuit and/or processor can determine the extent of tissue engagement. For example, if the in situ output signal strength is 60% of the output signal strength with no teeth/springs in contact with tissue, then the sensor extends into the tissue 40% of the total length of the sensor. For example, with ten teeth/springs total, the 60% output signal strength is the result of six teeth/spring not in contact with tissue; four teeth are in contact with tissue.

In the case of FIGS. 15, 16, and 17A, the output signal strength (e.g., acoustic pressure/intensity of the acoustic waves and/or amplitude of the electrical signal) is proportional to the number of teeth that are in contact with the tissue in a way that causes mechanical strain. Contact with the tissue (causing mechanical strain) can increase the output signal strength. For example, the processor can determine that the teeth that are in contact with the tissue are contributing most to the acoustic intensity and/or electrical signal and use the quantity of teeth that are in contact as a measure of how far the sensor extends into tissue. The processor and/or processor circuit can determine that the extent of tissue contact based on the number of teeth in contact with tissue because the output signal strength increases with increased tissue contact. For example, the sensor's total number of teeth/springs, the total length, and/or the output signal strength (e.g., in laboratory, testing, and/or experimental conditions) with none, all, or a portion of the teeth/springs in contact with tissue can be stored in a memory of the processor and/or processor circuit. Depending on the in situ (during the actual procedure) output signal strength, the processor circuit and/or processor can determine the extent of tissue engagement. For example, if the in situ output signal strength is 60% of the output signal strength with all teeth/springs in contact with tissue (causing mechanical strain), then the sensor extends into the tissue 60% of the total length of the sensor. For example, with ten teeth/springs total, the 60% output signal strength is the result of six teeth/spring in contact with tissue (causing mechanical strain); four teeth are not in contact with tissue.

Depending on the implementation, the system may be set up according to either FIGS. 4A-6 and 17B (tissue contact is proportional to number of teeth not in contact with tissue) or FIGS. 15, 16, and 17A (tissue contact is proportional to number of teeth in contact with tissue, causing mechanical strain). In some aspects, the system is not set up to accordingly to both FIGS. 4A-6, 17B and FIGS. 15, 16, 17A simultaneously.

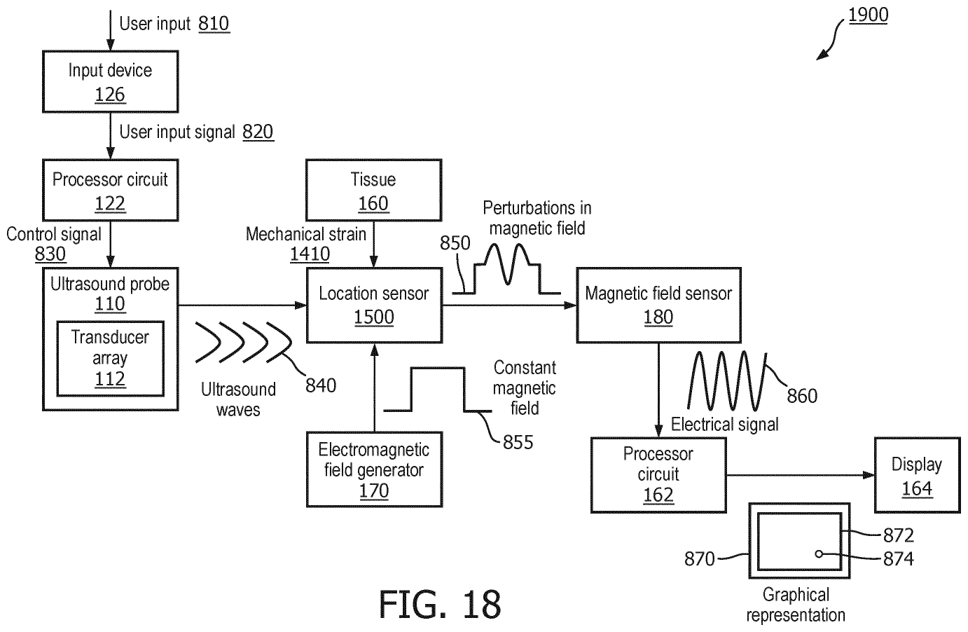

FIG. 18 is a schematic representation, in block diagram form, of at least a portion of the example instrument tracking system 1900 employing components of the tissue engagement sensing system 100, in accordance with at least one embodiment of the present disclosure. In the example shown in FIG. 19, similar to FIG. 8, above, the instrument tracking system 1900 is operated in reverse (e.g., relative to operation of the system 100 shown in FIG. 14) such that the ultrasound system is used to excite vibrations in the engagement sensor/location sensor 1500, generating an oscillating magnetic field which is detected by the magnetic field sensor 180 for purposes of tracking the position of the engagement sensor 1500, and therefore of the instrument to which it is affixed.

Unlike the tissue engagement sensor 150 of FIG. 8, the location sensor 1500 of FIG. 19 uses magneto-elastic springs 1530 (see FIG. 15) in place of teeth 420 and magneto-elastic members 430. These springs 1530 can experience mechanical strain 1410 when contacted by the tissue 160, and can thus generate larger perturbations 850 in the magnetic field 855, resulting in a larger electrical signal 860 being generated by the magnetic field sensor 180. For example, an amplitude of the perturbations 850 (e.g., oscillating magnetic field) is configured to increase in response to the increase in the magneto-strictive response caused by the mechanical strain 1410. Thus, the location of the tissue engagement sensor/location sensor 1500 of FIG. 19 may be easier to detect (e.g., with the magnetic field sensor 180 and processor circuit 162) and display (e.g., on the display 164) than the tissue engagement sensor/location sensor 150 of FIG. 8.

Accordingly, it can be seen that the tissue engagement sensing system advantageously provides a way by which tissue engagement by a medical device can be accurately measured in real time while a procedure is taking place. Furthermore, the same hardware can be used, either sequentially or in parallel, to track the position of the medical device within the patient's body.

A number of variations are possible on the examples and embodiments described above. For example, the teeth and support member could have a variety of shapes or cross sections different from the examples shown herein, while still performing the same or a similar function. The teeth may be cantilevered, or may be anchored at both ends. More than one tissue engagement sensor, with similar or different fundamental frequencies, may be used during the same procedure. In general, the sensor can include multiple discrete units/masses of magneto-restrictive material. The units/masses of magneto-restrictive material can be coupled to and/or otherwise arranged on a substrate or substrate segments. For example, the units/masses of magneto-restrictive material can be spaced from one another (e.g., by a fixed/constant distance). Tissue can cover a given quantity of the units/masses of magneto-restrictive material, depending on a depth that the sensor engages the tissue. Instead of cantilevered teeth, the sensor may use circular or elliptical discs in a 2D array configuration, similar to drum heads. Such a configuration may for example be analogous to a capacitive machined ultrasound transducer (CMUT) sensor that works on magnetic fields instead of sound waves. Because it is a MEMS device, the tissue engagement sensor may be detected visually, or based on its acoustic signature (e.g., a bright point on the surface of a medical instrument, emitting at a narrow range of frequencies).

The technology described herein may be employed for any medical procedure where accurate tissue engagement is desired, including intravascular procedures, intracardiac procedures, lead removal procedures, and other invasive procedures of the tissue or organs of the body. The tissue engagement sensing system may also have applications in non-medical industries, e.g., to determine the depth of penetration of a nail, pin, screw, or tool.

Accordingly, the logical operations making up the embodiments of the technology described herein are referred to variously as operations, steps, objects, elements, components, or modules. Furthermore, it should be understood that these may occur or be performed in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the tissue engagement sensing system. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the tissue engagement sensing system as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. A system, comprising:
a magnetic field subsystem configured to generate an oscillating magnetic field;
a magneto-elastically driven oscillator configured to, in the presence of the oscillating magnetic field, generate acoustic waves;
an ultrasound probe configured to receive the generated acoustic waves and generate an electrical signal indicative of the generated acoustic waves; and
a processing circuit configured to output, to a display, a graphical representation indicative of the generated acoustic waves,
wherein contact between the magneto-elastically driven oscillator and tissue of a patient causes a change in the generated acoustic waves, the generated electrical signal, and the graphical representation.

2. The system of claim 1, further comprising:
a second magneto-elastically driven oscillator configured to, in the presence of the oscillating magnetic field, generate the acoustic waves; and
a support member, wherein the magneto-elastically driven oscillator and the second magneto-elastically driven oscillator extend from the support member,
wherein at least one of a frequency or an amplitude of the generated acoustic waves is indicative of contact between the tissue and the magneto-elastically driven oscillator or the second magneto-elastically driven oscillator,
such that the graphical representation is indicative of a degree of engagement of the tissue by the magneto-elastically driven oscillator and the second magneto-elastically driven oscillator.

3. The system of claim 2, further comprising a tissue-engaging medical instrument,
wherein the support member is coupled to a tissue-engaging portion of the tissue-engaging instrument,
such that the graphical representation is indicative of a degree of engagement of the tissue by the tissue-engaging portion.

4. The system of claim 3, further comprising a plurality of magneto-elastically driven oscillators, wherein the plurality of magneto-elastically driven oscillators comprise the magneto-elastically driven oscillator,
wherein the graphical representation is indicative of a degree of engagement of the tissue by the tissue-engaging portion, and
wherein the processing circuit is configured to determine the degree of engagement based on a quantity of the plurality of magneto-elastically driven oscillators that are in contact with the tissue.

5. The system of claim 2, further comprising a plurality of additional magneto-elastically driven oscillators extending from the support member and configured to, in the presence of the oscillating magnetic field, generate the acoustic waves.

6. The system of claim 2,
wherein the support member comprises a first material, and
wherein each magneto-elastic oscillator comprises a tooth comprising the first material.

7. The system of claim 6, wherein each magneto-elastic oscillator further comprises a magneto-elastic material different from the first material.

8. The system of claim 2, further comprising a second support member, wherein a first end of each magneto-elastic oscillator is coupled to the support member and a second end of each magneto-elastic oscillator is coupled to the second support member.

9. The system of claim 1, wherein the magneto-elastic oscillator is configured to be at least partially immersed in a fluid, wherein the generated acoustic waves propagate through the fluid, and wherein the ultrasound probe is configured to receive the generated acoustic waves through at least the fluid.

10. The system of claim 1, wherein the ultrasound probe is further configured to generate second acoustic wave,
wherein the magneto-elastic oscillator is further configured to, in the presence of the second acoustic waves, generate a second oscillating magnetic field;
wherein the system further comprises a magnetic field subsystem configured to detect a location of the magneto-elastic oscillator associated with the second oscillating magnetic field and generate a corresponding location signal; and
a processing circuit configured to receive the location signal and output, to a display, a second graphical representation indicative of the location signal.

11. The system of claim 1, wherein the change in the generated acoustic waves comprises a decrease in an amplitude of the generated acoustic waves.

12. The system of claim 11, further comprising a plurality of magneto-elastically driven oscillators,
wherein the plurality of magneto-elastically drive oscillators comprise the magneto-elastically driven oscillator,
wherein the graphical representation is indicative of a degree of engagement with the tissue, and
wherein the processing circuit is configured to determine the degree of engagement based on a quantity of the plurality of magneto-elastically driven oscillators that are not in contact with the tissue.

13. The system of claim 1, wherein the magneto-elastically driven oscillator comprises a microelectromechanical systems (MEMS) spring.

14. The system of claim 1, wherein a change in the generated acoustic waves comprises an increase in an amplitude of the generated acoustic waves.

15. The system of claim 14, wherein a magneto-strictive response of the magneto-elastically driven oscillator is configured to increase in response to a mechanical strain caused by contact with the tissue.

16. A system, comprising:
an ultrasound probe configured to generate acoustic waves;
a magneto-elastically driven oscillator configured to, in the presence of the acoustic waves, generate an oscillating magnetic field;
a magnetic field subsystem configured to detect a location of the magneto-elastically driven oscillator associated with the oscillating magnetic field and generate a corresponding location signal; and
a processing circuit configured to receive the location signal and output, to a display, a first graphical representation indicative of the location signal.

17. The system of claim 16, further comprising a medical instrument, wherein the magneto-elastic oscillator is coupled to a portion of the medical instrument, such that the first graphical representation is indicative of a location of the portion.

18. The system of claim 16, further comprising:
a second magneto-elastically driven oscillator configured to, in the presence of the acoustic waves, generate the oscillating magnetic field; and
a support member, wherein the magneto-elastically driven oscillator and the second magneto-elastically driven oscillator extend from the support member.

19. The system of claim 18,
wherein at least one of a frequency or an amplitude of the oscillating magnetic field is indicative of contact between tissue and the magneto-elastically driven oscillator or the second magneto-elastically driven oscillator,
wherein the processing circuit is further configured to generate a second graphical representation indicative of a degree of engagement of the tissue by the magneto-elastically driven oscillator or the second magneto-elastically driven oscillator.

20. The system of claim 19, further comprising a tissue-engaging instrument,
wherein the support member is coupled to a tissue-engaging portion of the tissue-engaging instrument,
such that the second graphical representation is indicative of a degree of engagement of the tissue by the tissue-engaging portion.

21. The system of claim 20, further comprising a plurality of additional magneto-elastically driven oscillators extending from the support member and configured to, in the presence of the second oscillating magnetic field, generate the oscillating magnetic field.

22. The system of claim 21, wherein the support member comprises a first material, and wherein each magneto-elastic oscillator comprises a tooth comprising the first material.

23. The system of claim 22, wherein each magneto-elastic oscillator further comprises a magneto-elastic material different from the first material.

24. The system of claim 16, wherein a magneto-strictive response of the magneto-elastically driven oscillator is configured to increase in response to a mechanical strain caused by contact with tissue of a patient.

25. The system of claim 24, wherein an amplitude of the oscillating magnetic field is configured to increase in response to the increase in the magneto-strictive response.

26. The system of claim 16, wherein the magneto-elastically driven oscillator comprises a microelectromechanical systems (MEMS) spring.

27. A system, comprising:
a sensor comprising:
a support structure;
a plurality of teeth extending from the support structure; and
a plurality of corresponding magneto-elastic members coupled to the plurality of teeth,
wherein:
an excitation of the plurality of teeth by first acoustic waves causes the plurality of magneto-elastic members to generate a first oscillating magnetic field detectable by a magnetic field detector,
an excitation of the plurality of magneto-elastic members by a second oscillating magnetic field causes the plurality of teeth to generate second acoustic waves detectable by an ultrasound probe,
an engagement of one or more of the teeth by tissue of a patient changes at least one of an amplitude or a frequency of the first oscillating magnetic field and of the second acoustic waves by an amount corresponding to a number of the teeth that are engaged by the tissue.
